(19) United States
(12) Patent Application Publication
(10) Pub. No.: US 2023/0310357 A1
(43) Pub. Date: Oct. 5, 2023

(54) ANALGESIC AGENT

(71) Applicant: Medical Corporation Yuyukai, Tokyo (JP)

(72) Inventor: Yuji OKUNO, Tokyo (JP)

(21) Appl. No.: 18/041,415

(22) PCT Filed: Jul. 6, 2021
(86) PCT No.: PCT/JP2021/025464

§ 371 Date: Feb. 13, 2023

(30) Foreign Application Priority Data
Sep. 1, 2020 (JP) 2020-146957

(51) Int. Cl. A61K 31/407; A61L 24/00; A61K 31/198; A61P 29/00; A61K 9/14

(52) U.S. Cl. CPC A61K 31/198; A61K 9/14; A61K 31/407; A61L 24/001; A61P 29/00

(57) ABSTRACT

The present invention provides an analgesic means that works by embolizing abnormal vessels and can be performed more safely. Provided is an analgesic agent that includes a particulate embolic substance having an average particle size from 10-200 µm, inclusive, and is administered via a catheter distal to a branch of a spinal artery in an artery feeding a site of pain.

### TECHNICAL FIELD

The present invention relates to a therapeutic agent for pain.

### BACKGROUND ART

An intractable pain such as a stiff shoulder pain, a lower back pain, and a cervical pain has been known as one of the symptoms of aging.

A cause of such a pain has been unknown for many years, but the inventor has conducted extensive studies and found that abnormal blood flow due to an increase in abnormal capillary vessels (known as “problematic vessels” or “problem vessels”) contributes to the pain (Non-Patent Document 1). Such an abnormal capillary vessel is caused by nerve growth and the like. Based on this, the inventor found a treatment method by infusing an embolic material (imipenem cilastatin particles) into the abnormal capillary vessel using a catheter to thereby clog the abnormal capillary vessel and normalize blood flow (see, for example, Non-Patent Document 2).

Non-Patent Document 1: Nat Med. 2012 Aug;18(8):1208-16
Non-Patent Document 2: doi:https://doi.org/10.1016/j.jvir.2016.09.028

### DISCLOSURE OF THE INVENTION

### Problems to Be Solved by the Invention

However, there is a further need for technology that enables a safer method for treating a pain by embolizing an abnormal capillary vessel.

The present invention is made in view of circumstances mentioned above, and an object thereof is to provide a more safely practicable means for treating a pain by embolizing an abnormal capillary vessel.

### Means for Solving the Problems

The present inventor has found that the above problem can be solved by administering using a catheter an embolic material to a predetermined site in an artery, and thus the present invention has been completed. More specifically, the present invention provides the following.

(1) A therapeutic agent for pain, the agent including:
a particulate embolic material having an average particle diameter of 10 µm or more and 200 µm or less,
the agent being administered via a catheter distal to a branch of a spinal artery in an artery feeding a site of pain.

(2) The therapeutic agent for pain according to (1), wherein the pain is one or more selected from the group consisting of a chronic cervical pain, a stiff shoulder pain, a chronic lower back pain, sciatica, and post-herpetic neuralgia.

### Effects of the Invention

According to the present invention, provided is a more safely practicable means for treating a pain by embolizing an abnormal capillary vessel.

### PREFERRED MODE FOR CARRYING OUT THE INVENTION

Although embodiments of the present invention will be described hereafter, the present invention is not limited thereto.

### Therapeutic Agent for Pain

A therapeutic agent for pain according to the present invention (hereinafter also may be referred to as “therapeutic agent of the present invention”) includes a particulate embolic material having an average particle diameter of 10 µm or more and 200 µm or less and is administered via a catheter distal to a branch of a spinal artery in an artery feeding a site of pain.

As mentioned above, the present inventor has developed a treatment method by infusing an embolic material into an abnormal capillary vessel to thereby clog the abnormal capillary vessel and normalize blood flow. However, this method has a problem in ensuring safety due to various complications (e.g., a temporary severe pain in chest and bilateral forearms, temporary involuntary movement, temporary discomfort, a numbness symptom for days to weeks, a weakness symptom for days).

Therefore, as a result of further studies, the present inventor has found that a therapeutic effect for pain can be safely exerted without any complication by infusing an embolic material distal to a branch of a spinal artery.

The phrase “treating a pain” as used herein means relieving or fully eliminating a pain.

The term “pain” as used herein means any pain that occurs in a body, and a site of the pain or a disease that causes the pain is not particularly limited. The pain in the present invention may be those in which a branch of a spinal artery is present between a site accessible to an artery (a radial artery, an inguinal artery, a transverse cervical artery, a suprascapular artery, a circumflex scapular artery, an iliolumbar artery, a lateral sacral artery, a superior gluteal artery, an inferior gluteal artery, an obturator artery, a profunda femoris artery, a lateral genicular artery, an anterior tibial recurrent artery, a fibular artery, etc.) and an abnormal blood vessel-forming site which causes the pain and typically is a pain that occurs in, for example, neck, shoulders, lower back, back (post-herpetic neuralgia, etc.) and trunk. From the viewpoint that the therapeutic agent of the present invention is likely to be effective, the pain may be one or more selected from the group consisting of a chronic cervical pain, a stiff shoulder pain, a chronic lower back pain, sciatica, and post-herpetic neuralgia. A type of the pain can be identified by a physician’s diagnosis.

Hereinafter, a composition of the therapeutic agent of the present invention will be described.

### (Embolic Material)

A particulate material having an average particle diameter of 10 µm or more and 200 µm or less is used as an embolic material.

The phrase “embolic material” as used herein means a material capable of blocking blood flow in an artery.

An average particle diameter of the embolic material can be appropriately adjusted depending on a diameter of an artery to be embolized, etc.

A lower limit of the average particle diameter of the embolic material is preferably 2 µm or more and more preferably 5 µm or more from the viewpoint that the artery to be embolized is easily embolized.

An upper limit of the average particle diameter of the embolic material is preferably 100 µm or less, more preferably 50 µm or less, and further preferably 30 µm or less from the viewpoints of avoiding unintended clogging of an artery other than an abnormal capillary vessel or easiness of delivery to the artery to be embolized.

The “average particle diameter” as used herein means an average value of particle size distribution and is determined using a laser diffraction-type particle size distribution measuring device (e.g., “SALD” series, manufactured by SHIMADZU CORPORATION).

A shape of the embolic material is not particularly limited, but may be amorphous, spherical, rectangular, etc.

The embolic material is not particularly limited as long as it can block blood flow in an artery and does not adversely affect a living body. A material of the embolic material may be a material poorly soluble or insoluble in blood at a body temperature (e.g., 35 to 39° C.), in particular, imipenem cilastatin, etc. However, from the viewpoint of preventing a normal blood vessel from clogging when the embolic material unintentionally enters the normal blood vessel, the embolic material may also be a material temporarily poorly soluble in blood at a body temperature (e.g., 35 to 39° C.) but eventually soluble in the blood at the body temperature.

The embolic material may also be a commercially available material in use for endovascular embolization in the medical art. Examples of such a commercially available material include a spherical embolic material such as “HepaSphere (trademark)” (Merit Medical Systems, Inc.) and “DC Bead (trademark)” (Eisai Co., Ltd.). The “HepaSphere (trademark)” having a particle diameter of 30 to 200 µm is known (https://www. merit.com/peripheral-intervention/embolotherapy/hepatic-oncology/hepasphere-microspheres/#toggle-id-3). The “DC Bead (trademark)” having a particle diameter of 100 µm or more is known (https://medical. eisai.jp/products/DCB_V100/).

### (Form of Therapeutic Agent)

The therapeutic agent of the present invention is a preparation for intraarterial administration. Therefore, the therapeutic agent of the present invention may include, in addition to the embolic material, a medium that can be administered to a living body (physiological buffer, sterile water, saline, a medium, a contrast agent) and an optional component that may be included in the preparation for intraarterial administration if needed as long as an embolizing effect of the embolic material is not impaired.

Furthermore, when the therapeutic agent of the present invention is administered, angiography may be performed in order to identify a site of administration of the therapeutic agent. Moreover, a contrast agent may be used in combination with the embolic material of the present invention for the angiography. A type of the contrast agent is not particularly limited, but iohexol or the like may be used. Note that, the contrast agent may be included in the therapeutic agent of the present invention (that is, may be administered concurrently with the embolic material) or may be administered separately from the therapeutic agent of the present invention. When the contrast agent is administered separately from the therapeutic agent of the present invention, an order of administration is not particularly limited, but preferably the contrast agent is administered first from the viewpoint of easiness of identification of the site of administration. Furthermore, the contrast agent is preferably included in the therapeutic agent of the present invention since the presence or absence of unintentional entry of the embolic material into a spinal artery due to backflow, etc. can be visually presumed.

### (Catheter)

The therapeutic agent of the present invention is administered using a catheter.

A diameter or shape of the catheter is not particularly limited as long as it is a hollow tube capable of being inserted into an artery and infusing the embolic material. Usually, a catheter having an outer diameter of 0.6 to 1 mm (so-called microcatheter) may be suitably used.

For operation of the catheter, a device or a technology usually employed in administration with the catheter such as a guide wire or a pump may be used.

For administration through the catheter, injection or free-flow administration may be appropriately selected depending on the site of administration. For example, at a site at which arteries communicate with each other, the injection administration is preferred. In this case, it is desirably confirmed that a distance from a spinal artery (e.g., 2 cm or more) is kept. For example, the free flow administration is preferred from the viewpoint of reducing a risk of the embolic material flowing into the spinal artery due to backflow upon the injection.

In the present invention, the backflow upon administration through the catheter can be prevented by administering the embolic material to a site in which a contrast agent infused in advance is confirmed to spontaneously flow into a peripheral blood vessel (referred to as washout).

### (Site of Administration)

The therapeutic agent of the present invention is administered distal to a branch of a spinal artery in an artery feeding a site of pain.

As mentioned above, the present inventor has found a treatment method by infusing an embolic material into an abnormal capillary vessel to thereby clog the abnormal capillary vessel and normalize blood flow (Non-Patent Document 2). Although a method for administering the embolic material through a truncal artery including a spinal blood vessel is considered for administering the embolic material to the abnormal capillary vessel, this method has a safety problem such as a complication. However, a site of administration that allows a satisfactory therapeutic effect to be exerted while ensuring safety has been unknown.

Therefore, as a result of studies, the present inventor has found that the safety problem mentioned above is caused by unintentional entry of the infused embolic material into a blood vessel feeding a spinal artery or a blood vessel feeding a central nerve. The present inventor has further found that an effective treatment can be performed without impairing safety by selecting, as the site of administration, a site distal to a branch of a spinal artery rather than a spinal artery itself.

In the present invention, the “distal to a branch of a spinal artery in an artery feeding a site of pain” is identified as follows. A site distal to the thus-identified branch (far side from a heart) is set as the site of administration for the therapeutic agent of the present invention. First, a catheter is inserted into an artery feeding a site of pain in a trunk and a somatic artery (an artery feeding a cervical spine, a thoracic spine, a lumbar spine, etc.) is subjected to angiography. Based on visualization of the angiography, a position of a branch of a spinal artery (an anterior spinal artery and/or a posterior spinal artery) is identified. Based on the visualized position of the branch, a position beyond the branch distally is identified as distal to a branch of a spinal artery in an artery feeding a site of pain, and an opening of the catheter is placed at the thus-identified position. Thus, the therapeutic agent of the present invention can be administered. In this case, the site of administration is preferably away from the spinal artery to the extent that the spinal artery is not visualized.

When the therapeutic agent is administered distal to the branch of a spinal artery as identified above, the embolic material does not unintentionally enter the spinal cord due to high arterial pressure from the spinal artery even though the site of administration is potentially connected to an arterial network of the spinal cord. Therefore, according to the present invention, administration distal to the branch of a spinal artery in an artery feeding a site of pain allows an effective pain treatment by embolizing an abnormal capillary vessel to be more safely performed.

A distance between the branch of a spinal artery and the site of administration of the therapeutic agent of the present invention (opening of the catheter) (how distal from the branch of a spinal artery) is not particularly limited, but is preferably 10 mm or more in order to prevent the embolic material from flowing into the spinal artery due to backflow. On the other hand, if the distance is too far, the embolic material is difficult to administer when a distance between the branch and the abnormal blood vessel that causes a pain is short. Therefore, the distance may be 50 mm or less.

In the present invention, the therapeutic agent is preferably administered distal to all of blood vessels corresponding to the “branch of a spinal artery in an artery feeding a site of pain”. However, in the present invention, an aspect in which the therapeutic agent is administered proximal to a blood vessel that is not sufficiently visualized with a contrast agent (e.g., blood vessel having a diameter of less than 0.5 mm) is not excluded even though the blood vessel corresponds to the “branch of a spinal artery in an artery feeding a site of pain”. This is because an adverse effect for a patient is limited when administered proximal to such a narrow branched blood vessel.

A particular site of administration of the therapeutic agent of the present invention may be appropriately set depending on the site of pain. Hereinafter, a method for identifying the site of administration will be exemplified for each particular pain. Note that, various arteries which are sites of administration exemplified below include branches branching from the arteries.

### [Chronic Cervical Pain]

When the pain is a chronic cervical pain, a catheter is inserted from a site accessible to an artery (a radial artery, an inguinal artery, etc.), advanced to a subclavian artery, and appropriately subjected to angiography at an origin of a costocervical artery. A microcatheter is advanced to a deep cervical artery and a highest intercostal artery avoiding a branch of an anterior spinal artery feeding a spinal cord. If blood flow to a site of pain is confirmed, that site may be set as a site of administration. However, when the deep cervical artery is less developed, it may not feed the site of the cervical pain. In such a case, a branch feeding a cervix is branched distal to an ascending cervical artery which is a branch of a thyrocervical trunk and the microcatheter is inserted into the branch, which may be set as the site of administration. In addition thereto, a transverse cervical artery may also be set as the site of administration.

### [Stiff Shoulder Pain (Pain Around Scapula)]

When the pain is a stiff shoulder pain, a catheter is inserted from a site accessible to an artery (a radial artery, an inguinal artery, etc.), advanced to a subclavian artery, and subjected to angiography at an origin of a costocervical artery. A microcatheter is advanced to a deep cervical artery and a highest intercostal artery avoiding a branch of an anterior spinal artery feeding a spinal cord. If blood flow to a site of pain is confirmed, that site may be set as a site of administration. In addition thereto, a transverse cervical artery, a suprascapular artery, and a circumflex scapular artery may also be set as the site of administration.

### [Chronic Lower Back Pain]

When the pain is a chronic lower back pain, a catheter is inserted from a site accessible to an artery (an inguinal artery, etc.), is advanced to an abdominal aorta, and subjected to angiography on each side at first to fourth lumbar arteries. In addition to the first to fourth lumbar arteries, an iliolumbar artery, a median sacral artery, a lateral sacral artery, and a superior gluteal artery may also be set as the site of administration avoiding a branch of a spinal branch feeding a spinal cord.

### [Sciatica]

When the pain is sciatica, a catheter is inserted from a site accessible to an artery (an inguinal artery, etc.), is advanced to an abdominal aorta, and subjected to angiography on each side at a fourth lumbar artery. A trunk may also be set as the site of administration avoiding a branch of a spinal branch feeding a spinal cord. In addition thereto, an iliolumbar artery, a lateral sacral artery, a superior gluteal artery, an inferior gluteal artery, an obturator artery, a profunda femoris artery, a lateral genicular artery, an anterior tibial recurrent artery, and a fibular artery may also be set as the site of administration.

### [Post-Herpetic Neuralgia]

When the pain is post-herpetic neuralgia, a catheter is inserted from a site accessible to an artery (an inguinal artery, etc.) and an artery feeding the site of pain is subjected to angiography. A somatic artery (a lumbar artery, an intercostal artery) may be set as the site of administration avoiding a branch of a spinal branch feeding a spinal cord.

### (Dosage, Etc.)

A dosage or a frequency of administration of the therapeutic agent of the present invention may be appropriately set depending on a condition of a patient (age, body weight, severity of a symptom, etc.).

### Therapeutic Effect of Therapeutic Agent of Present Invention

The therapeutic agent of the present invention allows a pain treatment by embolizing an abnormal capillary vessel to be performed safer. For example, the therapeutic agent of the present invention can treat a pain without various complications (e.g., a temporary severe pain in chest and bilateral forearms, temporary involuntary movement, temporary discomfort, a numbness symptom for days to weeks, a weakness symptom for days, etc.) which have been a concern in the conventional method for embolizing an abnormal capillary vessel.

A therapeutic effect of the therapeutic agent of the present invention can be evaluated by any method for evaluating a pain. Examples thereof include a method described in Example (method based on the “numerical rating scale (NRS) score”) and a method in which a patient subjectively answers the degree of pain relief (“Patients Global Impression of Change”).

### Therapeutic Method With Therapeutic Agent of Present Invention

The present invention encompasses a method for treating a pain with the therapeutic agent of the present invention.

A subject to be treated by the method for treating a pain of the present invention is not particularly limited, but encompasses human, non-human animals, etc.

A dosage or a number of times of administration of the particulate embolic material in the method for treating a pain of the present invention may be appropriately set depending on a condition of a subject (age, height, body weight, a degree of pain).

### Use of Particulate Embolic Material

The present invention encompasses a use of a particulate embolic material having an average particle diameter of 10 µm or more and 200 µm or less for producing the therapeutic agent for pain. In the use, the particulate embolic material is administered via a catheter distal to a branch of a spinal artery in an artery feeding a site of pain.

### EXAMPLES

Hereinafter, the present invention will be described with reference to Examples in more detail, but the present invention is not limited to these Examples.

### Preparation of Material, Etc.

In this Example, the following materials were used.

### (Embolic Material)

In this Example, imipenem cilastatin (product name “Primaxin”, manufactured by Merck & Co., Inc.) was used as an embolic material. This embolic material had an average particle diameter of 70 µm or less as measured by a laser diffraction-type particle size distribution measuring device (“SALD” series, manufactured by SHIMADZU CORPORATION).

### (Catheter)

In this Example, an angiographic catheter (outer diameter: 1 mm, manufactured by Medikit co. ltd.) and a microcatheter (outer diameter: 0.6 mm, manufactured by ASAHI INTECC CO., LTD.) were used.

### (Contrast Agent)

In this Example, Iopark (manufactured by Fuji Pharma Co., Ltd.) was used. The contrast agent was mixed with the embolic material. A mixing ratio (mass ratio of active ingredients) was set at 0.5 g of the embolic material relative to 10 mL of the contrast agent.

### Method for Administering Embolic Material

The embolic material was intravascularly administered to patients having any of the below-mentioned pains using a catheter. The embolic material was administered as the embolic material alone or as a mixed solution of the embolic material with the contrast agent. Note that, the embolic material was prepared as an aqueous solution of 0.5 g / 10 mL and administered so as to give a total dosage of 0.5 g to 2 g per patient as an amount of the embolic material.

### Chronic Cervical Pain

Forty-two patients having a chronic cervical pain received the embolic material. First, a catheter was inserted from a site accessible to an artery (a radial artery, an inguinal artery, etc.), advanced to a subclavian artery, and subjected to angiography at an origin of a costocervical artery. A microcatheter was advanced to a deep cervical artery and a highest intercostal artery avoiding a branch of an anterior spinal artery feeding a spinal cord and the embolic material was carefully administered by free flow so as to prevent backflow to the anterior spinal artery while blood flow to a site of pain was monitored. However, when the deep cervical artery is less developed, it may not feed a site of the cervical pain. In such a case, a branch feeding a cervix is branched distal to an ascending cervical artery which is a branch of a thyrocervical trunk, the microcatheter was inserted into the branch and the embolic material was administered thereto. Furthermore, a catheter was also inserted into a transverse cervical artery and the embolic material was administered thereto. In this Example, the number of times of administration of the embolic material was set to 1.

### Stiff Shoulder Pain (Pain Around Scapula)

Forty-two patients having a stiff shoulder pain received the embolic material. First, a catheter was inserted from a site accessible to an artery (a radial artery, an inguinal artery, etc.), advanced to a subclavian artery, and subjected to angiography at an origin of a costocervical artery. A microcatheter was advanced to a deep cervical artery and a highest intercostal artery avoiding a branch of an anterior spinal artery feeding a spinal cord and a mixed solution of the embolic material with the contrast agent was carefully administered by free flow so as to prevent backflow to the anterior spinal artery while blood flow to a site of pain was monitored. Furthermore, a catheter was also inserted into each of a transverse cervical artery, a suprascapular artery, and a circumflex scapular artery, and the mixed solution was administered thereto in a similar manner. In this Example, the number of times of administration of the embolic material was set to 1.

### Chronic Lower Back Pain

Thirteen patients having a chronic lower back pain received the embolic material. First, a catheter was inserted from a site accessible to an artery (an inguinal artery, etc.), advanced to the abdominal aorta, and subjected to angiography on each side at first to fourth lumbar arteries. A mixed solution of the embolic material with the contrast agent was administered to right and left lumber arteries by free flow avoiding a branch of a spinal branch feeding a spinal cord. A catheter was also inserted into each of a median sacral artery, a lateral sacral artery, and a superior gluteal artery, and the mixed solution was administered thereto in a similar manner. In this Example, the number of times of administration of the embolic material was set to 1.

### Sciatica

Ten patients having sciatica received the embolic material. First, a catheter was inserted from a site accessible to an artery (an inguinal artery, etc.), advanced to an abdominal aorta, and subjected to angiography on each side at a fourth lumbar artery. The embolic material was administered to a trunk by free flow avoiding a branch of a spinal branch feeding a spinal cord. Furthermore, a catheter was also inserted into each of an iliolumbar artery, a lateral sacral artery, a superior gluteal artery, an inferior gluteal artery, an obturator artery, a profunda femoris artery, a lateral genicular artery, an anterior tibial recurrent artery, and a fibular artery and the embolic material was administered thereto in a similar manner. In this Example, the number of times of administration of the embolic material was set to 1.

### Post-Herpetic Neuralgia

Three patients having post-herpetic neuralgia received the embolic material. First, a catheter was inserted from a site accessible to an artery (an inguinal artery, etc.), and an artery feeding a site of pain was subjected to angiography. A mixed solution of the embolic material with the contrast agent was administered to an artery feeding a site of pain by free flow avoiding a branch of a spinal branch feeding a spinal cord. In this Example, the number of times of administration of the embolic material was set to 1.

### Method for Evaluating Therapeutic Effect

After administration of the embolic material, a therapeutic effect in each patient was evaluated as follows.

In this Example, the “numerical rating scale (NRS) score”, one of common evaluation criteria of pain, was employed as an evaluation criterion for the therapeutic effect. In the score, the pain is evaluated based on post-treatment pain answered as an integer value (0 or more 10 or less) where pre-treatment pain was determined as “10”. Note that, the lower the value answered, the greater the post-treatment pain was relieved.

### Results

For the patients having a chronic cervical pain, 70% or more of the patients reported a score of 5 or less at 2 months after the end of the administration period. For the patients having a stiff shoulder pain, 70% or more of the patients reported a score of 5 or less at 2 months after the end of the administration period. For the patients having a chronic lower back pain, 70% or more of the patients reported a score of 5 or less at 3 months after the end of the administration period. For the patients having sciatica, 80% or more of the patients reported a score of 5 or less at 2 months after the end of the administration period. For the patients having post-herpetic neuralgia, all of the patients reported a score of 7 or less at 3 months after the end of the administration period.

As mentioned above, it was observed that the therapeutic agent of the present invention significantly ameliorated the pain. Furthermore, any adverse event (e.g., complication) due to the therapeutic agent of the present invention was not observed after the administration period, confirming safety of the therapeutic agent of the present invention.

Note that, for the patients having a chronic cervical pain, although the pain was observed to be relieved by administering the embolic material to the spinal artery as described above, complication (numbness) occurred.

1. A method of treatment for pain of a subject in need thereof, the method comprising:
administering to the subject an agent comprising a particulate embolic material having an average particle diameter of 10 µm or more and 200 µm or less,
the agent being administered via a catheter distal to a branch of a spinal artery in an artery feeding a site of pain.

2. The method of treatment for pain according to claim 1, wherein the pain is one or more selected from the group consisting of a chronic cervical pain, a stiff shoulder pain, a chronic lower back pain, sciatica, and post-herpetic neuralgia.
